(19) United States
(12) Patent Application Publication
(10) Pub. No.: US 2024/0008967 A1
(43) Pub. Date: Jan. 11, 2024

(54) Method of Electrochemically Boosting Tooth Whitening

(71) Applicant: Colgate-Palmolive Company, New York, NY (US)

(72) Inventors: Nihal DOGU, Dayton, NJ (US); Leighton DAVIES-SMITH, Lebanon, NJ (US); Hallena STROTMAN, Somerset, NJ (US)

(73) Assignee: Colgate-Palmolive Company, New York, NY (US)

(21) Appl. No.: 18/042,932

(22) PCT Filed: Aug. 24, 2021
(86) PCT No.: PCT/US2021/047241

§ 371 Date: Feb. 24, 2023

Related U.S. Application Data
(60) Provisional application No. 63/072,370, filed on Aug. 31, 2020.

(51) Int. Cl. A61C 19/06; A61K 8/22; A61Q 11/00

(52) U.S. Cl. CPC A61C 19/066; A61K 8/22; A61Q 11/00; A61K 2800/92

(57) ABSTRACT

A process for electrochemically whitening a tooth, the process including: a) contacting the tooth with a whitening gel having a first pH, the whitening gel located in a dental device comprising a positive electrode and a negative electrode; b) flowing a current between the positive electrode and the negative electrode through whitening gel to whitening the tooth such that the whitening gel transitions from the first pH to a second pH; c) irradiating the tooth with light emitted from a light source present on the dental device; wherein the whitening gel comprises a peroxide source; an electrolyte source; and wherein the second pH is greater than the first pH.

### CROSS-REFERENCE TO RELATED APPLICATIONS

The present application claims the benefit of priority to U.S. Provisional Patent Application Ser. No. 63/072,370, filed Aug. 31, 2020, the entirety of which is incorporated herein by reference.

### BACKGROUND

Tooth whitening processes include contacting a tooth surface with a whitening gel that comprises a peroxide source. However, previous attempts at achieving desirable tooth whitening performance often raised difficulties in the amount of peroxide source, the pH of such whitening agents, and whether a light is necessary to irradiate the tooth during such whitening process. Thus, a need exists for a new whitening process that overcomes such the limitations.

### BRIEF SUMMARY

The present invention includes a process for electrochemically whitening a tooth, the process comprising: a) contacting the tooth with a whitening gel having a first pH, the whitening gel located in a dental device comprising a positive electrode and a negative electrode; b) flowing a current between the positive electrode and the negative electrode through whitening gel to whitening the tooth such that the whitening gel transitions from the first pH to a second pH; wherein the whitening gel comprises a peroxide source present in an amount ranging from about 0.05 wt. % to about 15 wt. % based on the total weight of the whitening gel, and wherein the second pH is greater than the first pH.

Other embodiments of the present invention include a process of electrochemically boosting tooth whitening performance of a whitening gel, the process comprising: a) contacting a tooth with the whitening gel comprising a peroxide source, the whitening gel having a first pH; b) flowing a current between the positive electrode and the negative electrode through whitening gel such that the whitening gel electrochemically transitions from the first pH to a second pH; c) irradiating the tooth with light from a light source having a wavelength ranging from about 390 nm to about 430 nm; wherein the second pH and the first pH are different, and wherein steps b) and c) at least partially overlap.

Other embodiments of the present invention include a kit for tooth-whitening, the kit comprising: a dental device comprising: a light source configured to emit a light having a wavelength ranging from about 390 nm to about 430 nm; and a trough having a positive electrode and a negative electrode; a whitening gel having a first pH ranging from about 5 to about 6, the whitening gel comprising a peroxide source; an electrolyte source; and wherein whitening gel is configured to undergo an electrochemical change in pH from the first pH to a second pH, the second pH being greater than the first pH.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION

The following description of the preferred embodiment(s) is merely exemplary in nature and is in no way intended to limit the invention, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

The description of illustrative embodiments according to principles of the present invention is intended to be read in connection with the accompanying drawings, which are to be considered part of the entire written description. In the description of embodiments of the invention disclosed herein, any reference to direction or orientation is merely intended for convenience of description and is not intended in any way to limit the scope of the present invention. Relative terms such as “lower,” “upper,” “horizontal,” “vertical,” “above,” “below,” “up,” “down,” “top,” and “bottom” as well as derivatives thereof (e.g., “horizontally,” “downwardly,” “upwardly,” etc.) should be construed to refer to the orientation as then described or as shown in the drawing under discussion. These relative terms are for convenience of description only and do not require that the apparatus be constructed or operated in a particular orientation unless explicitly indicated as such.

Terms such as “attached,” “affixed,” “connected,” “coupled,” “interconnected,” and similar refer to a relationship wherein structures are secured or attached to one another either directly or indirectly through intervening structures, as well as both movable or rigid attachments or relationships, unless expressly described otherwise. Moreover, the features and benefits of the invention are illustrated by reference to the exemplified embodiments. Accordingly, the invention expressly should not be limited to such exemplary embodiments illustrating some possible non-limiting combination of features that may exist alone or in other combinations of features; the scope of the invention being defined by the claims appended hereto.

Unless otherwise specified, all percentages and amounts expressed herein and elsewhere in the specification should be understood to refer to percentages by weight. The amounts given are based on the active weight of the material. According to the present application, the term “about” means+/−5% of the reference value. According to the present application, the term “substantially free” less than about 0.1 wt. % based on the total of the referenced value.

The present invention is directed to a whitening gel, a kit comprising the whitening gel and a dental device, and a method of electrochemically boosting the whitening performance of the whitening gel.

The whitening gel of the present invention may be conductive to be suitable for an electrochemical whitening process. The whitening gel may comprise a bleaching agent, a thickener composition, and a source of electrolyte. The whitening gel of the present invention may further comprise a liquid carrier. The whitening gel of the present invention may further comprise a humectant. The whitening gel of the present invention may further comprise a structuring agent.

The bleaching agent may comprise a peroxide source. Non-limiting examples of the peroxide source may include hydrogen peroxide, urea peroxide, glyceryl peroxide, benzoyl peroxide, and combinations thereof.

The bleaching agent may be present in an amount ranging from about 0.1 wt. % to about 18.0 wt. %—including all amounts and sub-ranges there-between—based on the total weight of the whitening gel. In some embodiments, the bleaching agent may be present in an amount ranging from about 0.1 wt. % to about 10.0 wt. %—including all amounts and sub-ranges there-between—based on the total weight of the whitening gel. In some embodiments, the bleaching agent may be present in an amount of about 0.1 wt. %—based on the total weight of the whitening gel. In some embodiments, the bleaching agent may be present in an amount of about 1.0 wt. %—based on the total weight of the whitening gel. In some embodiments, the bleaching agent may be present in an amount of about 3.0 wt. %—based on the total weight of the whitening gel. In some embodiments, the bleaching agent may be present in an amount of about 6.0 wt. %—based on the total weight of the whitening gel. In some embodiments, the bleaching agent may be present in an amount of about 9.0 wt. %—based on the total weight of the whitening gel.

The whitening gel of the present invention may be suitable for an electrochemical tooth whitening process. As such, the whitening gel of the present invention may exhibit a level of electric conductivity that allows electric current to flow through the whitening gel, thereby activating the bleaching agent and accelerating the overall tooth whitening process. To exhibit suitable electrical conductivity, the whitening gel of the present invention may comprise an electrolyte source capable of conducting ions.

The electrolyte source may comprise one or more conductive salts. Conductive salts may be selected from one or more of inorganic salts and organic salts. Non-limiting examples of conductive salts include chloride salts (such as sodium chloride, potassium chloride, lithium chloride, calcium chloride, strontium chloride, magnesium chloride or other chloride salts. Non-limiting examples of other salts include sodium, potassium, lithium, calcium magnesium, strontium, fluoride, iodide, bromide. Non-limiting examples of potassium salts include water soluble potassium salt including potassium nitrate, potassium citrate, potassium chloride, potassium bicarbonate and potassium oxalate.

The electrolyte source may be present in an amount ranging from about 0.1 wt. % to about 8.0 wt. %—including all amounts and subranges there-between—based on the total weight of the whitening gel. The electrolyte source may be present in an amount ranging from about 1.0 wt. % to about 4.0 wt. %—including all amounts and subranges there-between—based on the total weight of the whitening gel.

The whitening gel of the present invention may have an electrical conductivity ranging from about 10.0 mS/cm to about 80.0 mS/cm—including all electrical conductivities and subranges there-between. In some embodiments, the whitening gel of the present invention may have an electrical conductivity ranging from about 55 mS/cm to about 85 mS/cm—including all electrical conductivities and subranges there-between. In some embodiments, the whitening gel of the present invention may have an electrical conductivity ranging from about 70 mS/cm to about 80 mS/cm—including all electrical conductivities and subranges there-between.

The whitening gel of the present invention may further comprise a liquid carrier. Non-limiting examples of liquid carrier include water. The water of the present invention may be deionized water, distilled water, or purified water.

The liquid carrier may be present in an amount ranging from about 65.0 wt. % to about wt. %—including all amounts and subranges there-between—based on the total weight of the whitening gel. In some embodiments, the liquid carrier may be present in an amount ranging from about 70.0 wt. % to about 80.0 wt. %—including all amounts and subranges there-between—based on the total weight of the whitening gel. In some embodiments, the liquid carrier may be present in an amount of about 78.0 wt. %, based on the total weight of the whitening gel.

The whitening gel of the present invention may further comprise a humectant. Non-limiting examples of humectant include polyol compounds. Examples of humectants include glycerin, sorbitol propylene glycol, xylitol, lactitol, polypropylene glycol, polyethylene glycol, hydrogenated corn syrup, and mixtures thereof.

The humectant may be present in an amount ranging from about 1.0 wt. % to about 9.0 wt. %—including all amounts and subranges there-between—based on the total weight of the whitening gel. In some embodiments, the humectant may be present in an amount ranging from about 2.0 wt. % to about 8.0 wt. %—including all amounts and subranges there-between—based on the total weight of the whitening gel. In some embodiments, the humectant may be present in an amount ranging from about 3.0 wt. % to about 7.0 wt. %—including all amounts and subranges there-between—based on the total weight of the whitening gel. In some embodiments, the humectant may be present in an amount ranging from about 4.0 wt. % to about 6.0 wt. %—including all amounts and subranges there-between—based on the total weight of the whitening gel. In some embodiments, the humectant may be present in an amount of about 5.0 wt. % based on the total weight of the whitening gel.

The whitening gel of the present invention may further comprise a structuring agent. A structuring agent may act to keep any solid phase of the whitening gel suspended, thus preventing separation of the solid phase portion of the oral care component from the liquid phase portion. Additionally, the structuring agent can provide body to the oral care composition.

The structuring agent of the present invention may be a non-ionic compound or component. Non-limiting examples of structuring agent include cellulose ether, xanthan gum, carrageenan, hydroxypropyl methylcellulose, hydroxyethyl cellulose, guar gum, tragacanth gum, karaya gum, arabic gum, starch, and combinations thereof. In a preferred embodiment, the structuring agent is hydroxyethyl cellulose.

The structuring agent may be present in an amount ranging from about 0.1 wt. % to about 5.0 wt. %—including all amounts and subranges there-between—based on the total weight of the whitening gel. In some embodiments, the structuring agent may be present in an amount ranging from about 1.0 wt. % to about 4.0 wt. %—including all amounts and subranges there-between—based on the total weight of the whitening gel. In some embodiments, the structuring agent may be present in an amount ranging from about 2.0 wt. % to about 3.0 wt. %—including all amounts and subranges there-between—based on the total weight of the whitening gel. In some embodiments, the structuring agent may be present in an amount of about 2.5 wt. % based on the total weight of the whitening gel.

The whitening gel of the present invention may comprise one or more surfactants. Non-limiting examples of surfactants include compositions that may be anionic, non-ionic, amphoteric, cationic and mixtures thereof. The surfactant may be present in an amount ranging from about 0.5 wt. % to about 5.0 wt.—including all amounts and sub-ranges there-between—based on the total weight of the whitening gel.

Non-limiting examples of anionic Surfactants include: Sulfonates and Sulfates: Suitable anionic surfactants include sulfonates and sulfates such as alkyl sulfates, alkylether sulfates, alkyl sulfonates, alkylether sulfonates, alkylbenzene sulfonates, alkylbenzene ether sulfates, alkylsulfoacetates, secondary alkane sulfonates, secondary alkylsulfates, alkyl sulfosuccinates and the like. Further, examples of anionic surfactants include water-soluble salts of higher fatty acid monoglyceride monosulfates, such as the sodium salt of the monosulfated monoglyceride of hydrogenated coconut oil fatty acids, higher alkyl sulfates such as sodium lauryl sulfate, alkyl aryl sulfonates such as sodium dodecyl benzene sulfonate, higher alkyl sulfoacetates, higher fatty acid esters of 1,2-dihydroxy propane sulfonate, and the substantially saturated higher aliphatic acyl amides of lower aliphatic amino carboxylic acid compounds, such as those having 12 to 16 carbons in the fatty acid, alkyl or acyl radicals, and the like. Phosphates and Phosponates: Suitable anionic surfactants also include phosphates such as alkyl phosphates, alkylether phosphates, aralkylphosphates, and aralkylether phosphates. Examples include a mixture of mono-, di- and tri-(alkyltetraglycolether)-o-phosphoric acid esters generally referred to as trilaureth-4-phosphate.

Non-limiting examples of amphoteric surfactants include surfactants having tertiary amine groups which may be protonated as well as quaternary amine containing zwitterionic surfactants. Those that may be useful include: Ammonium Carboxylate Amphoterics: Examples of such amphoteric surfactants include, but are not limited to: certain betaines such as cocobetaine and cocamidopropyl betaine; monoacetates such as sodium lauroamphoacetate; diacetates such as disodium lauroamphoacetate; amino- and alkylamino-propionates such as lauraminopropionic acid

Non-limiting examples of nonionic surfactants include: Polyethylene oxide extended sorbitan monoalkylates (i.e., Polysorbates); Polyalkoxylated alkanols, such as polyethoxylated octyl or nonyl phenols having HLB values of at least about 14. Sulfated and phosphated derivatives of these surfactants may also be useful. Examples of such derivatives include ammonium nonoxynol-4-sulfate. Surfactants based on block copolymers of ethylene oxide (E0) and propylene oxide (PO) may also be suitable. Polyalkoxylated glycols such as ethylene glycol, propylene glycol, glycerol, and the like may be partially or completely esterified, i.e., one or more alcohols may be esterified, with a (C8 to C22) alkyl carboxylic acid.

Non-limiting examples of cationic Surfactants include but are not limited to primary amines, secondary amines, tertiary amines, quaternary amines, alkanolamines, mono-alkyl alkanolamines, di-alkyl alkanolamines, tri-alkyl alkanolamines, alkyl mono alkanolamines, alkyl di-alkanolamines, alkylamines, mono-alkyl amines, di-alkyl amines, tri-alkylamines, alkoxylated amines, alkyl and aryl amine alkoxylates, methoxylated alkylamines, ethoxylated alkylamines, alkoxylated alkanolamines, alkyl alkanolamines, alkoxylated ethylene diamine derivatives, alkyl/aryl/arylalkyl amine oxides.

The whitening gel of the present invention may further comprise one or more flavorant. Non-limiting examples of flavorant include wintergreen. The flavorant may be present in an amount ranging from about 0.1 wt. % to about 0.5 wt. %—including all amounts and subranges there-between—based on the total weight of the whitening gel. In some embodiments, the flavorant may be present in an amount of about 0.3 wt. % based on the total weight of the whitening gel.

The whitening gel of the present may further comprise one or more buffers. Non-limiting examples of buffers include primary, secondary, or tertiary alkali metal phosphates, citric acid, sodium citrate, sodium saccharin, tetrasodium pyrophosphate, sodium hydroxide, and combinations thereof.

The buffers may be present in an amount ranging from about 0.1 wt. % to about 1.5 wt. %—including all amounts and subranges there-between—based on the total weight of the whitening gel. In some embodiments, the buffers may be present in an amount of about 0.2 wt. % to about 1.1 wt. % based on the total weight of the whitening gel.

As discussed, the present invention includes an electrochemical tooth whitening process that includes flowing an electrical current through the whitening gel, thereby creating a pH shift in the whitening gel. Surprisingly, it has been surprisingly discovered that the electrochemical pH shift in the whitening gel imparts a boost in whitening performance of the whitening gel on a tooth surface. As a result, enhanced tooth whitening may be achieved without necessitating increased amounts of bleaching agent or excessive numbers of treatment cycles.

With the present invention including a pH shift, the whitening gel may exhibit a first pH and a second pH, whereby the whitening gel electrochemically shifts from the first pH to the second pH upon a current being applied to the whitening gel.

The current applied to the whitening gel may be from a low-voltage DC power source—whereby the voltage ranges from about 1.0 VDC to about 6.0 VDC—including all voltages and subranges there-between. The current applied to the whitening gel may ranges from about 10.0 mA to about 40.0 mA—including all voltages and subranges there-between.

The first pH of the whitening gel may be the pH value of the whitening gel without electrical current applied to the whitening gel. The first pH may be the pH value of the whitening gel as provided to the user from the manufacturer. The first pH of the whitening gel may range from about 4.5 to about 6.5—including all pH values and subranges there-between. In a preferred embodiment, the first pH of the whitening gel may range from about 5.0 to about 6.0—including all pH values and subranges there-between. In some embodiments, the first pH is acidic. In some embodiments, the first pH is neutral.

The second pH of the whitening gel may be the pH value of the whitening gel during the application of electrical current during the electrochemical whitening process. The second pH of the whitening gel may range from about 7.0 to about 11.0—including all pH values and subranges there-between. In some embodiments, the second pH of the whitening gel may range from about 7.0 to about 10.0—including all pH values and subranges there-between. In some embodiments, the second pH of the whitening gel may range from about 8.0 to about 10.5—including all pH values and subranges there-between.

In some embodiments, the second pH of the whitening gel may be about 7.5. In some embodiments, the second pH of the whitening gel may be about 8.0. In some embodiments, the second pH of the whitening gel may be about 9.0. In some embodiments, the second pH of the whitening gel may be about 9.5. In some embodiments, the second pH may be neutral. In some embodiments, the second pH may be alkaline (basic).

In a non-limiting embodiment, the whitening gel may have a first pH ranging from about to about 6.0—including all pH values and subranges there-between. In such embodiments, the bleaching agent may comprise a peroxide source that is present in an amount ranging from about 8.0 wt. % to about 10.0 wt. % based on the total weight of the whitening gel. In such embodiments, the peroxide source may be present in an amount of about 9.0 wt. % based on the total weight of the whitening gel. In such embodiments, the second pH may range from about 7.0 to about 8.0—including all pH values and subranges there-between.

In a non-limiting embodiment, the whitening gel may have a first pH ranging from about to about 6.0—including all pH values and subranges there-between. In such embodiments, the bleaching agent may comprise a peroxide source that is present in an amount ranging from about 5.0 wt. % to about 7.0 wt. % based on the total weight of the whitening gel. In such embodiments, the peroxide source may be present in an amount of about 6.0 wt. % based on the total weight of the whitening gel. In such embodiments, the second pH may range from about 7.0 to about 8.0—including all pH values and subranges there-between. In such embodiments, the second pH may be about 7.5.

In a non-limiting embodiment, the whitening gel may have a first pH ranging from about to about 6.0—including all pH values and subranges there-between. In such embodiments, the bleaching agent may comprise a peroxide source that is present in an amount ranging from about 2.0 wt. % to about 4.0 wt. % based on the total weight of the whitening gel. In such embodiments, the peroxide source may be present in an amount of about 4.0 wt. % based on the total weight of the whitening gel. In such embodiments, the second pH may range from about 7.0 to about 10.0—including all pH values and subranges there-between. In such embodiments, the second pH may be about 7.5.

In a non-limiting embodiment, the whitening gel may have a first pH ranging from about to about 6.0—including all pH values and subranges there-between. In such embodiments, the bleaching agent may comprise a peroxide source that is present in an amount ranging from about 0.05 wt. % to about 0.2 wt. % based on the total weight of the whitening gel. In such embodiments, the peroxide source may be present in an amount of about 0.1 wt. % based on the total weight of the whitening gel. In such embodiments, the second pH may range from about 8.0 to about 10.5—including all pH values and subranges there-between. In such embodiments, the second pH may range from about 8.0 to about 10.0—including all pH value and subranges there-between.

The electrochemical process of the present invention may further include irradiating a tooth surface with light emitted from a light source during the electrochemical whitening process. The light may have a wavelength ranging from about 390 nm to about 430 nm—including all wavelengths and subranges there-between. In some embodiments, the light may have a wavelength of about 410 nm.

The electrochemical process of the present invention includes first contacting a tooth with the whitening gel and applying a current to the whitening gel. The electrochemical process may further comprise irradiating the tooth with the light in a step that at least partially overlaps with the application of current to the whitening gel. As such, the electrochemical process of the present invention provides that a tooth may be irradiated by light having a wavelength ranging from about 390 nm to about 430 nm—including all wavelengths and subranges there-between—while the whitening gel has a pH value that is equal to the second pH.

In some embodiments, the whitening gel may be located in a dental device, whereby the dental device comprises a positive electrode and a negative electrode configured to apply the current to the whitening gel. The whitening gel may be in direct contact with both of the positive electrode and the negative electrode in the dental device. The dental device may further comprise the light source that is configured to emit light at a wavelength ranging from about 390 nm to about 430 nm—including all wavelengths and subranges there-between.

The whitening process of the present invention may comprise a plurality of treatment cycles—whereby each treatment cycle comprises applying current to the whitening gel to shift the pH value from the first pH to the second pH for a treatment period. Each treatment cycle may further comprise irradiating the tooth with the light emitted from the light source for at least a portion of the treatment period. The treatment period may span a first period of time ranging from about 5 seconds to about 360 seconds—including all times and subranges there-between. Each treatment period may be spaced apart by a non-treatment period that includes no contact of the whitening gel to the tooth surface, no electrochemical shift in pH of the whitening gel. The non-treatment period may span a second period of time that is greater than the treatment period.

The present invention further comprises a kit that includes both the whitening gel and the dental device. The dental device may comprise a tray. The tray may comprise a trough. The positive electrode and the negative electrode may be at least partially located within the trough. The whitening gel may be located within the trough. During use, at least one tooth is positioned in the trough such that the tooth directly contacts the whitening gel. The light source may be positioned within the dental device such that light emitted by the light source is directed into the trough and is incident on a tooth located in such trough of the tray of the dental device.

According to the present invention, the whitening gel may be pre-applied to the dental device. In other embodiments, the whitening gel may be supplied in a separate container having a reservoir containing the whitening gel, whereby the user applied the whitening gel to the dental device at the time of tooth whitening. The kit may further comprise a power source electrically coupled to the positive electrode and the negative electrode. In other embodiments, the kit may be configured for the positive electrode and the negative electrode to be electrically connected to a power source. The kit may further comprise a power source electrically coupled to the light source. In other embodiments, the kit may be configured for the light source to be electrically connected to a power source.

Non-limiting examples of a power source include a battery or an electrical socket. According to the embodiments where the dental device is configured to be electrically coupled to a wall socket, the dental device may further comprise an AC/DC power transformer.

### Examples

The following includes a number of experiments that were performed to test the unexpected boost in tooth whitening after electrochemically shifting the pH of whitening gel. The experiments include a number of treatment cycles, whereby a tooth surface was contacted with a whitening gel formulation having a first pH, whereby the whitening gel comprises a bleaching agent. After each treatment cycle, whitening effect imparted to the tooth was recorded by measuring the change in color value of the corresponding tooth.

The experiment tested the effects of pH shift—whereby at least some of the formulations were electrochemically shifted from the first pH to a second pH as set forth in each respective tables. For the examples not listing a second pH, those corresponding whitening gel formulations did not undergo an electrochemical pH shift during the treatment cycle—rather such formulations were maintained at the first pH during the treatment cycle. Other parameters tested included irradiating the tooth surface with light having a wavelength of 410 nm during the treatment cycle, as well as applying external heat to the tooth surface during the treatment cycle.

Experiment 1

A first experiment was performed by testing a whitening gel formulation having a peroxide whitening agent in a concentration of about 9.0 wt. % and a first pH between 5 and 6. After each treatment, the color change imparted on the tooth surface was measured and recorded—as set forth below in Table 1.

TABLE 1

| | Peroxide | Second | | | T1 | T2 | T3 | T4 | T5 |
|---|---|---|---|---|---|---|---|---|---|
| | wt. % | pH | Light | Heat | ΔE | ΔE | ΔE | ΔE | ΔE |
| Ex. 1 | 9.0 | — | Yes | No | −5.0 | −9.4 | −11.4 | −12.6 | −13.7 |
| Ex. 2 | 9.0 | 7.0 | Yes | No | −8.5 | −13.0 | −15.3 | −16.1 | −16.6 |
| Ex. 3 | 9.0 | 7.5 | Yes | No | −8.8 | −14.2 | −15.4 | −15.9 | −16.8 |
| Ex. 4 | 9.0 | 8.0 | Yes | No | −9.5 | −13.0 | −14.1 | −14.6 | −15.3 |
| Ex. 5 | 9.0 | 9.0 | Yes | No | −8.5 | −9.4 | −9.3 | −9.3 | −9.0 |

As demonstrated by Table 1, an unexpected boost in tooth whitening performance occurs when a 9.0 wt. % peroxide whitening gel has pH value shifted to a second pH ranging from about 7.0-8.0.

Experiment 2

A second experiment was performed by testing another whitening gel formulation having a peroxide whitening agent in a concentration of about 9.0 wt. % and a first pH between 5 and 6. According to the second experiment, no electrochemical shift occurred while the application of light and/or heat was varied. After each treatment, the color change imparted on the tooth surface was measured and recorded—as set forth below in Table 2.

TABLE 2

| | Peroxide | Second | | | T1 | T2 | T3 | T4 | T5 |
|---|---|---|---|---|---|---|---|---|---|
| | wt. % | pH | Light | Heat | ΔE | ΔE | ΔE | ΔE | ΔE |
| Ex. 6 | 9.0 | — | No | No | −5.0 | −9.4 | −11.4 | −12.6 | −13.7 |
| Ex. 7 | 9.0 | — | Yes | No | −6.7 | −10.8 | −13.4 | −13.8 | −14.4 |
| Ex. 8 | 9.0 | — | No | Yes | −2.8 | −4.6 | −5.5 | −7.4 | −8.5 |
| Ex. 9 | 9.0 | — | Yes | Yes | −9.7 | −13.8 | −15.0 | −16.0 | −17.5 |

As demonstrated by Table 2, specifically, Example 7, without the electrochemical shift from the first pH to the second pH, the application of light or heat fails to produce the same whitening efficacy that is achieved after a shift from the first pH to the second pH. Furthermore, as demonstrated by Example 9, a whitening gel having 9.0 wt. % peroxide which undergoes an electrochemical pH shift can surprisingly exhibit in tooth-whitening effect that is comparable to a whitening boost achieved when both light and heat are applied without such pH shift. Therefore, comparing Examples 2-4 against Example 9—the present invention further provides a surprising benefit of imparting superior whitening efficacy without needing any heating during the treatment cycle—thereby protecting a user's oral cavity.

Experiment 3

A third experiment was performed by testing a whitening gel formulation having a peroxide whitening agent in a concentration of about 0.1 wt. % and a first pH between 5 and 6. After each treatment, the color change imparted on the tooth surface was measured and recorded—as set forth below in Table 3.

TABLE 3

| | Peroxide | Second | | | T1 | T2 | T3 | T4 | T5 |
|---|---|---|---|---|---|---|---|---|---|
| | wt. % | pH | Light | Heat | ΔE | ΔE | ΔE | ΔE | ΔE |
| Ex. | 0.1 | — | Yes | No | −0.6 | −1.8 | −3.2 | −4.5 | −5.9 |
| 10 | | | | | | | | | |
| Ex. | 0.1 | 8.0 | Yes | No | −1.5 | −3.2 | −4.4 | −5.0 | −6.0 |
| 11 | | | | | | | | | |
| Ex. | 0.1 | 9.5 | Yes | No | −1.8 | −4.6 | −5.7 | −7.0 | −8.1 |
| 12 | | | | | | | | | |
| Ex. | 0.1 | 10.5 | Yes | No | −1.3 | −4.5 | −6.3 | −8.0 | −9.0 |
| 13 | | | | | | | | | |
| Ex. | 0.1 | 9.0 | No | No | −0.8 | −1.6 | −2.4 | −3.1 | −3.7 |
| 14 | | | | | | | | | |
| Ex. | 0.1 | 10.5 | No | No | −1.2 | −2.4 | −3.2 | −4.2 | −4.8 |
| 15 | | | | | | | | | |
| Ex. | 0.1 | — | Yes | Yes | −1.7 | −3.1 | −4.8 | −6.2 | −8.5 |
| 16 | | | | | | | | | |
| Ex. | 0.1 | — | No | Yes | −0.6 | −0.7 | −0.6 | −1.4 | −1.3 |
| 17 | | | | | | | | | |

As demonstrated by Examples 10-14 of Table 3, an unexpected boost in tooth whitening performance occurs when a 0.1 wt. % whitening gel has pH value shifted to a second pH value that ranges from about 8.0 to about 10.5. In particular, Examples 11-13 show a marked boost in whitening efficacy when the pH of the whitening gel is electrochemically shifted to a second pH ranging from 8.0-10.5 as compared to the whitening performance of Example 10. Furthermore, Examples 14 and 15 demonstrate that improved whitening performance can even be achieved in the absence of light when the whitening gel has been electrochemically shifted to the second pH (compare Example 10 vs. Examples 14 and 15).

Moreover, Example 16 demonstrates that, at 0.1 wt. % peroxide concentration, the electrochemical pH shift can surprisingly exhibit in tooth-whitening effect that is comparable to a whitening boost achieved when both light and heat are applied without such pH shift. Therefore, the present invention further provides a surprising benefit of imparting superior whitening efficacy without needed any heating during the treatment cycle—thereby protecting a user's oral cavity.

Experiment 4

A fourth experiment was performed by testing a whitening gel formulation having a peroxide whitening agent in a concentration of about 3.0 wt. % and a first pH between 5 and 6. After each treatment, the color change imparted on the tooth surface was measured and recorded—as set forth below in Table 4.

TABLE 4

| | Peroxide | Second | | | T1 | T2 | T3 | T4 | T5 |
|---|---|---|---|---|---|---|---|---|---|
| | wt. % | pH | Light | Heat | ΔE | ΔE | ΔE | ΔE | ΔE |
| Ex. | 3.0 | — | No | No | −3.0 | −6.0 | −8.0 | −9.0 | −10.0 |
| 18 | | | | | | | | | |
| Ex. | 3.0 | — | Yes | No | −5.3 | −9.8 | −13.3 | −14.4 | −15.3 |
| 19 | | | | | | | | | |
| Ex. | 3.0 | 7.5 | Yes | No | −5.9 | −10.7 | −13.1 | −14.7 | −15.4 |
| 20 | | | | | | | | | |
| Ex. | 3.0 | 8.5 | Yes | No | −5.4 | −10.4 | −12.4 | −14.0 | −14.9 |
| 21 | | | | | | | | | |
| Ex. | 3.0 | 9.5 | Yes | No | −5.3 | −9.4 | −10.6 | −13.1 | −14.1 |
| 22 | | | | | | | | | |
| Ex. | 3.0 | 9.9 | Yes | No | −9.4 | −13.9 | −14.3 | −15.3 | −15.7 |
| 23 | | | | | | | | | |
| Ex. | 3.0 | 9.9 | No | No | −4.9 | −7.8 | −9.3 | −10.7 | −12.1 |
| 24 | | | | | | | | | |
| Ex. | 3.0 | 9.9 | Yes | Yes | −5.0 | −9.1 | −10.9 | −12.1 | −12.8 |
| 25 | | | | | | | | | |

As demonstrated by Example 23 of Table 4, an unexpected boost in tooth whitening performance occurs when a 3.0 wt. % whitening gel has pH value shifted to a second pH value that ranges from about 10.0 in the presence of light (Example 23). Example 20 also demonstrates an unexpected boost in whitening performance at a second pH of 7.5 at a peroxide concentration of 3.0 wt. %.

Experiment 5

A fifth experiment was performed utilizing a whitening gel formulation having a peroxide whitening agent in varying concentrations, wherein each formulation has a first pH between 5 and 6. After each treatment, the color change imparted on the tooth surface was measured and recorded—as set forth below in Table 5.

TABLE 5

| | Peroxide | Second | | | T1 | T2 | T3 | T4 | T5 |
|---|---|---|---|---|---|---|---|---|---|
| | wt. % | pH | Light | Heat | ΔE | ΔE | ΔE | ΔE | ΔE |
| Ex. | 1.0 | 9.1 | Yes | No | −4.5 | −8.0 | −10.9 | −12.6 | −13.1 |
| 26 | | | | | | | | | |
| Ex. | 1.0 | 9.1 | No | No | −1.3 | −2.5 | −4.0 | −4.6 | −5.4 |
| 27 | | | | | | | | | |
| Ex. | 1.5 | 9.9 | No | No | −4.7 | −7.0 | −9.0 | −10.1 | −11.4 |
| 28 | | | | | | | | | |
| Ex. | 1.5 | 9.9 | Yes | No | −7.7 | −11.2 | −12.5 | −13.1 | −13.3 |
| 29 | | | | | | | | | |
| Ex. | 6.0 | 7.5 | Yes | No | −8.2 | −11.1 | −14.4 | −15.5 | −15.9 |
| 30 | | | | | | | | | |

As also demonstrated by Table 5, as demonstrated by Table 5, an unexpected boost in tooth whitening performance occurs for both whitening compositions at 1.0 wt. % peroxide (Example 26) and 1.5 wt. % peroxide with in the presence of light (Example 29).

1. A process for electrochemically whitening a tooth, the process comprising:
a) contacting the tooth with a whitening gel having a first pH, the whitening gel located in a dental device comprising a positive electrode and a negative electrode;
b) flowing a current between the positive electrode and the negative electrode through the whitening gel to whiten the tooth such that the whitening gel transitions from the first pH to a second pH;
wherein the whitening gel comprises a peroxide source present in an amount ranging from about 0.05 wt. % to about 15 wt. % based on the total weight of the whitening gel, and wherein the second pH is greater than the first pH.

2. The process according to claim 1, wherein the process further comprises c) irradiating the tooth with light having a wavelength ranging from about 390 nm to about 430 nm.

3. The process according to claim 2, wherein the wavelength of the light is about 410 nm.

4. The process according to claim 1, wherein the first pH ranges from about 5 to about 6.

5. The process according to claim 1, wherein the peroxide source is present in an amount ranging from about 8.0 wt. % to about 10.0 wt. % based on the total weight of the whitening gel and the second pH ranges from about 7.0 to about 8.0.

6. (canceled)

7. The process according to claim 1, wherein the peroxide source is present in an amount ranging from about 5.0 wt. % to about 7.0 wt. % based on the total weight of the whitening gel and the second pH is about 7.5.

8. (canceled)

9. The process according to claim 1, wherein the peroxide source is present in an amount ranging from about 2.0 wt. % to about 4.0 wt. % based on the total weight of the whitening gel and the second pH ranges from about 7.0 to about 10.0.

10. (canceled)

11. The process according to claim 1, wherein the peroxide source is present in an amount ranging from about 0.5 wt. % to about 2.0 wt. % based on the total weight of the whitening gel and the second pH ranges from about 9.0 to about 10.0.

12. (canceled)

13. The process according to claim 1, wherein the peroxide source is present in an amount ranging from about 0.05 wt. % to about 0.2 wt. % based on the total weight of the whitening gel and the second pH ranges from about 8.0 to about 10.5.

14. (canceled)

15. The process according to claim 1, wherein the peroxide source is selected from the group consisting of hydrogen peroxide, urea peroxide, glyceryl peroxide, benzoyl peroxide.

16. (canceled)

17. (canceled)

18. A process of electrochemically boosting tooth whitening performance of a whitening gel, the process comprising:
a) contacting a tooth with the whitening gel comprising a peroxide source, the whitening gel having a first pH;
b) flowing a current between the positive electrode and the negative electrode through whitening gel such that the whitening gel electrochemically transitions from the first pH to a second pH; and
c) irradiating the tooth with light from a light source having a wavelength ranging from about 390 nm to about 430 nm;
wherein the second pH and the first pH are different, and wherein steps b) and c) at least partially overlap.

19. The process according to claim 18, wherein the wavelength of the light is about 410 nm.

20. The process according to claim 18, wherein the first pH ranges from about 5 to about 6.

21. The process according to claim 18, wherein the peroxide source is present in an amount ranging from about 8.0 wt. % to about 10.0 wt. % based on the total weight of the whitening gel and the second pH ranges from about 7.0 to about 8.0.

22. (canceled)

23. The process according to claim 18, wherein the peroxide source is present in an amount ranging from about 5.0 wt. % to about 7.0 wt. % based on the total weight of the whitening gel and the second pH is about 7.5.

24. (canceled)

25. The process according to claim 18, wherein the peroxide source is present in an amount ranging from about 2.0 wt. % to about 4.0 wt. % based on the total weight of the whitening gel and the second pH ranges from about 7.0 to about 10.0.

26. (canceled)

27. The process according to claim 18, wherein the peroxide source is present in an amount ranging from about 0.5 wt. % to about 2.0 wt. % based on the total weight of the whitening gel and the second pH ranges from about 9.0 to about 10.0.

28. (canceled)

29. The process according to claim 18, wherein the peroxide source is present in an amount ranging from about 0.05 wt. % to about 0.2 wt. % based on the total weight of the whitening gel and the second pH ranges from about 8.0 to about 10.5.

30. (canceled)

31. The process according to claim 18, wherein the peroxide source is selected from the group consisting of hydrogen peroxide, urea peroxide, glyceryl peroxide, benzoyl peroxide, and wherein the whitening gel further comprises water.

32. (canceled)

33. A kit for tooth-whitening, the kit comprising:
a dental device comprising:
a light source configured to emit a light having a wavelength ranging from about 390 nm to about 430 nm; and
a trough having a positive electrode and a negative electrode;
a whitening gel having a first pH ranging from about 5 to about 6, the whitening gel comprising:
a peroxide source; and
an electrolyte source;
wherein the whitening gel is configured to undergo an electrochemical change in pH from the first pH to a second pH, the second pH being greater than the first pH.

34.-47. (canceled)
